(19) United States
(12) Patent Application Publication
(10) Pub. No.: US 2004/0138919 A1
(43) Pub. Date: Jul. 15, 2004

(54) Method and system for wound care assessment and treatment

(76) Inventor: Morris Edward Gales III, Southfield, MI (US)

Correspondence Address: JOHN S. ARTZ, ARTZ & ARTZ, P.C., SUITE 250, 28333 TELEGRAPH ROAD, SOUTHFIELD, MI, 48034, US

(21) Appl. No.: 10/339,521

(22) Filed: Jan. 9, 2003

(51) Int. Cl. G06F 17/60; A61N 1/00

(52) U.S. Cl. 705/2; 607/88

(57) ABSTRACT

A skin care surveillance system includes a tool which assists a staff member in examining a patient and allowing for the recordation of certain items relevant to the examination of the patient. The tool contains a plurality of triggers to look for by the staff member while the examination is being performed. The tool includes an area associated with each of the plurality of triggers to allow for the recordation of any steps taken in response to an identification of one or more of the plurality of triggers. The tool also contains an identification area for the identity and shift of the staff member performing the examination as well as the date the examination is performed.

The present invention relates generally to the prevention and treatment of skin wounds. More particularly, the present invention relates to a method and system for minimizing the occurrence and improving the treatment of skin wounds of patients.

### BACKGROUND OF THE INVENTION

It is well understood that individuals who are residents in long care facilities—not to mention their families—expect excellent care from the staff who work at such facilities. Obviously, no one expects to be harmed due to neglect of the staff at these facilities. In fact, state and federal governmental regulatory agencies exist and have developed regulations to ensure that such neglect and harm to these residents does not occur. Unfortunately, despite these regulations and agencies, such harm and neglect still occurs. These state and federal regulatory bodies are therefore charged with the difficult assignment or task of identifying the existence or occurrence of neglect and harm. Further, in instances where neglect and harm have been identified, these bodies are responsible for determining the appropriate consequences for a person who was responsible for such neglect or the facility where such neglect occurred. Moreover, these regulatory bodies are also responsible for informing patients and their families or guardians if a negative outcome in a patient was due to neglect or if neglect contributed to a deterioration of the patient's overall condition. Alternatively, these bodies can determine whether a negative outcome was due to patient noncompliance.

It is well known that many patients that are admitted to long care facilities are in the late stages of human life with multiple health problems. Skin related problems or skin wounds are known to be relatively prevalent in these types of patients as compared to the remainder of the population. This is partly because of the frailty of these people who are typically elderly as well as their typical lack of mobility. As a result, these elderly, frail and/or non-mobile patients are susceptible to skin related problems. These problems include wounds due to lack of movement, as a result of being moved, or by falling or bumping into something. Moreover, any skin related problems may be the result of a systemic progressive decline rather then neglect on the part of the caregiver.

Even if a skin related injury is determined to be caused by neglect, it is often difficult to determine who is responsible for the neglect. This is partly because it is difficult to track who was informed of the existence of the wound on the patient, whether appropriate action was taken and, if not, who failed to take the appropriate action. In view of a recently stated governmental goal of zero tolerance for long term care facility acquired wounds, it would be desirable to provide a system and method for accurately detecting the existence of skin wounds as well as for providing treatment therefore. It would also be desirable to provide a system and method for accurate responsibility assessment in the event a skin related injury is caused by neglect.

Current wound assessment records and procedures for evaluating the same are currently utilized. However, these records and procedures are relatively complex and difficult to analyze. Moreover, the current records and procedures focus primarily on the wounds themselves and treatment thereof. They do not focus on any measures to prevent the occurrence of new wounds. Accordingly, it is presently almost impossible to assign any responsibility for neglect such that training and/or discipline can be effectively accomplished.

### SUMMARY OF THE PRESENT INVENTION

Accordingly, it is an object of the present invention to provide a skin care surveillance system that maximizes skin wound prevention and thereby decreases the risk of skin wounds in patients.

It is another object of the present invention to provide a skin care surveillance system that improves the treatment of skin wounds in the event that a patient develops one or more such wounds.

It is a further object of the present invention to provide a skin care surveillance system that assists in identifying the cause of a skin wound in the event that a patient develops one or more such wounds.

It is still another object of the present invention to provide a skin care surveillance system that assists in identifying the person or persons responsible for a skin wound in the event it is caused by neglect of a caregiver or other avoidable circumstance.

It is still a further object of the present invention to provide a skin care surveillance system that is relatively simple to use.

It is yet another object of the present invention to provide a skin care surveillance system that reduces the time associated with evaluating, treating, and analyzing a skin wound and thus yields significant financial savings.

It is yet a further object of the present invention to provide a skin care surveillance system that allows for the determination of whether a facility is overstaffed, understaffed, or properly staffed.

In accordance with the above and other objects of the present invention, a skin care surveillance system is provided. The skin care surveillance system includes a first tool to assist a staff member or direct care giver in examining a patient and allowing for the recordation of certain items relevant to the examination. The first tool includes a plurality of questions that are to be answered by a direct caregiver. The questions assist in identifying the existence of any new skin wounds as well as determining whether certain triggers or conditions exist with respect to the patient that could cause new skin wounds to develop. The first tool also requires the direct caregiver to execute the form verifying that the information about their analysis of the patient's skin and factors that can affect the occurrence of wounds is accurate. The first tool further includes a location for execution by the direct caregiver's supervisor after the supervisor has reviewed the information on the first tool. This assists in identifying the supervisor who was given the data related to each patient, to assure accountability for information transfer in the event any patient neglect has occurred.

The system also includes a second tool that is intended to be completed by the supervisor. The second tool includes a list with a plurality of triggers that the supervisor is alerted to look for during their examination of the patient. The second tool also includes a location thereon which is associated with each of the plurality of triggers to allow for the recordation of any remedial steps taken in response to an identification of a new wound or one or more of the plurality of triggers. The second tool further contains an identification area for the identity and the shift of the supervisor performing the examination as well as the date the examination was performed to assure accountability for proper patient care.

These and other features and advantages of the present invention will become apparent from the following description of the invention, when viewed in accordance with the accompanying drawings and appended claims.

### BRIEF DESCRIPTION OF THE DRAWINGS

FIG. 1 is a flow chart illustrating the skin care surveillance system in accordance with a preferred embodiment of the present invention;

FIG. 2 is a schematic illustration of a first skin care surveillance system tool in accordance with one embodiment of the present invention;

FIG. 3 is a schematic illustration of another portion of the first skin care surveillance system tool shown in FIG. 2;

FIG. 4 is a schematic illustration of a second skin care surveillance system tool in accordance with one embodiment of the present invention; and

FIG. 5 is a schematic illustration of a skin care surveillance system tool in accordance with another embodiment of the present invention.

### DETAILED DESCRIPTION OF THE PREFERRED EMBODIMENTS

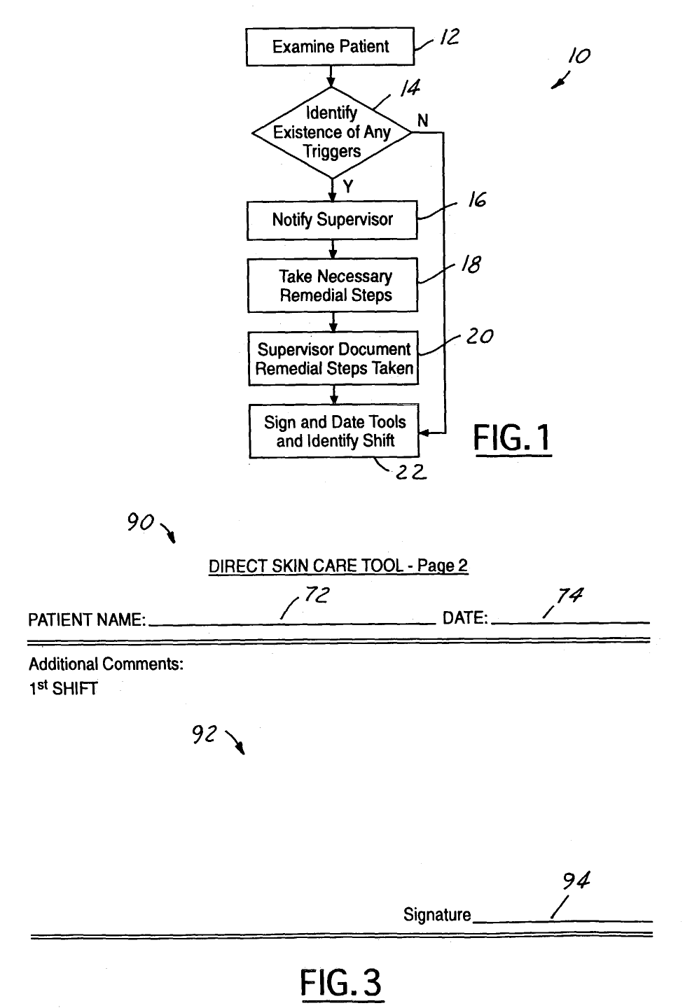

Referring now to FIG. 1, which illustrates a skin care surveillance system in accordance with the present invention. The skin care surveillance system 10 is intended to minimize the occurrence of skin wounds as well as to provide immediate care in the event a skin wound develops. The skin care surveillance system 10 also assists in providing accountability and assigning responsibility in the event a wound develops due to neglect or if a wound is mistreated. In one embodiment, the skin care surveillance system 10 is intended for use in facilities where long-term care is provided, such as nursing homes or locations for assisted living because it is in these facilities where skin wounds in patients are typically most prevalent. However, it should be understood that the skin care surveillance system 10 can be utilized in any location where a skin wound can occur and where patients are being treated, such as in any hospital or doctor's office.

As shown, in one embodiment of the skin care surveillance system 10, the first step is for the staff member responsible for a given patient to perform an examination of the patient, as generally indicated by reference number 12. As used herein, the term staff member can refer to a nurse or other person responsible for monitoring the patient. As is known, patient records are kept in association with each patient such that the patient records can be reviewed by subsequent shifts or any other person providing care to the patient, such as the staff member's supervisor or the person ultimately responsible for the care of the patient, such as the doctor.

During the examination process, the staff member reviewing the patient is trained to look for any new skin wounds or any triggers that could cause a skin wound to develop, as generally indicated by reference number 14. If no new wounds or triggers—which are conditions that are known to be a potential cause for a new wound—are present, then the staff member notes the appropriate information in the patient's chart. In one embodiment, the relevant information includes the staff member's name, the date and time the examination occurred and the shift of the staff member. This information is thus available for review by the supervisor or other responsible person.

If a new wound or a trigger is detected or identified as being present during the examination of the patient, as generally indicated by reference number 14, the staff member records the existence of this condition, such as by noting this in the patient's file. If a new wound or trigger is identified, the staff member then notifies their supervisor, as generally indicated by reference number 16. Once the supervisor is notified, it is determined what—if any—remedial actions should be taken. Thereafter, either the supervisor or the staff member under the supervisor's control or supervision will take any necessary remedial steps, as generally indicated by reference number 18. If remedial actions are taken, the supervisor will document these steps in the patient's file or record, as generally indicated by reference number 20.

If no remedial steps are taken, this will also be recorded in the patient record. Once any necessary remedial steps are taken and documented, the supervisor notes the appropriate information in the patient's chart and then signs and dates the documentation as well as indicating the shift in which any steps were taken, as generally indicated by reference number 22. Similarly, if a patient examination is completed, which does not reveal the existence of any new wounds or any triggers, this information is noted in the patient's chart. Thereafter, the patient's chart is signed and dated, as generally indicated by reference number 22, such that this information can be subsequently reviewed.

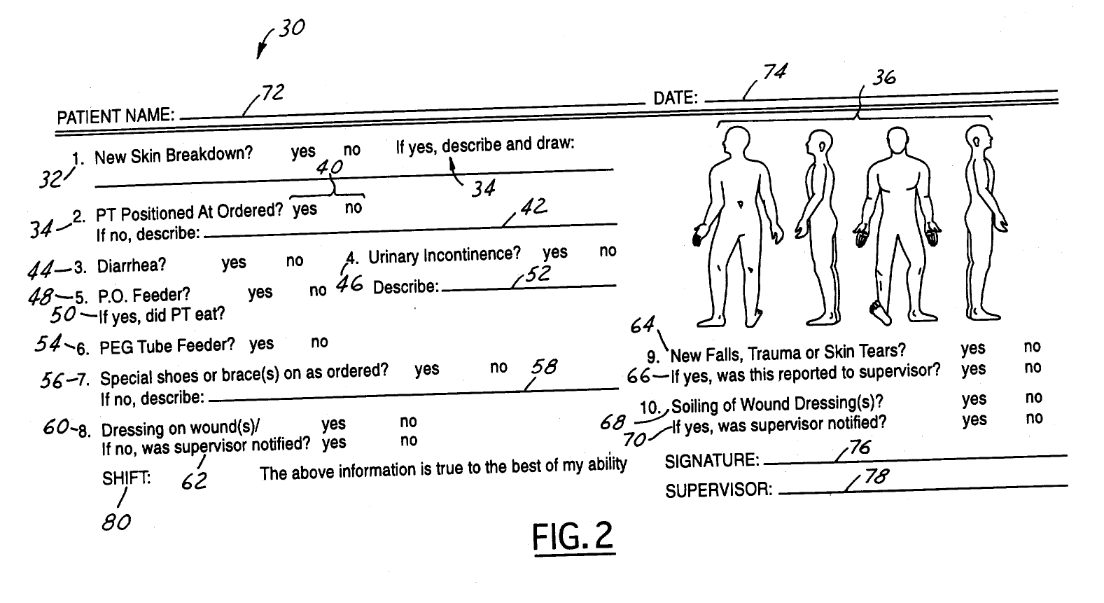

Referring now to FIGS. 2 and 3, which illustrate an exemplary tool 30 for use in connection with the skin care surveillance system 10. The exemplary tool 30, in one embodiment, is the first or primary tool in the skin care surveillance system 10 and is intended for use during the examination of the patient, such as discussed above in connection with step 12. As discussed below, the tool 30 allows for the identification of trends and potential problem areas on patients to be quickly and easily identified. It will be appreciated that other tools or resources may be needed or may be utilized as desired.

As discussed above, the tool 30 is preferably filled out by a staff member or other person charged with routinely examining the patient. The exemplary tool 30 preferably includes a series of questions that help the staff member in evaluating the existence of any triggers, as discussed above in connection with step 14. For example, the first question, generally indicated by reference number 32, asks the staff member to determine whether a “new skin breakdown” has occurred. The staff member can then check or circle the appropriate response. If the answer to this question is “yes”, the staff member should describe the new wound with the description being placed in the area generally indicated by reference number 34. The staff member should also draw the location and/or configuration of the wound on the human representations, generally indicated by reference number 36. It will be understood that the configurations of the human representations may differ.

The second question seeks to determine whether the patient has been positioned as previously ordered, as generally indicated by reference number 38. The staff member should indicate the appropriate response, i.e. “yes” or “no”, as generally indicated by reference number 40. If the answer to the second question is “no”, the patient's improper position should be described in the area generally indicated by reference number 42. The third question seeks to determine if the patient had an onset of diarrhea, as generally indicated by reference number 44. This question again preferably requires the staff member to answer the question by circling either a “yes” or “no”. The fourth question seeks to determine if the patient was suffering from urinary incontinence, as generally indicated by reference number 46. This question also seeks a “yes” or “no” response from the staff member. The fifth question asks whether the patient is a per oral (P.O.) feeder, as generally indicated by reference number 48. If the answer to the above is yes, i.e. that the patient did have a P.O. feeder, the staff member is asked whether the patient did eat, as generally indicated by reference number 50. If the patient did eat, i.e. the answer to the above question is “yes”, the staff member should describe the percentage of food offered that the patient ate, as generally indicated by reference number 52.

The sixth question asks whether the patient has a Gastrostomy Tube (PEG feeder), as generally indicated by reference number 54. Again, this question seeks a “yes” or “no” response from the staff member. The seventh question, as generally indicated by reference number 56, requires the staff member to identify if the patient was wearing special shoes or brace as required. If the answer to the above question is no, the details should be described in the area indicated by reference number 58. The eighth question asks whether the dressing is on the patient's wound, as generally indicated by reference number 60. If the answer to the above question is no, the staff member should indicate whether the supervisor was notified of the removal of the dressing, as generally indicated by reference number 62.

Obviously, if the patient does not have any wounds that require dressing, the eighth question is not applicable and should be skipped. The ninth question, as generally indicated by reference number 64, asks whether the patient experienced any new falls, trauma or skin tears. If the answer is yes, the staff member must indicate whether this fact was reported to the supervisor, as generally indicated by reference number 66. The tenth question, as generally indicated by reference number 68, asks whether the wound dressing was soiled. If the answer is yes, the staff member must indicate whether this fact was reported to the supervisor, as generally indicated by reference number 70. Again, if the patient did not have any wounds which needed dressing, the tenth question does not apply and should be skipped.

It will be understood that the questions contained on the primary tool 30 are intended to identify any new wounds that may have developed on a patient or any conditions or triggers that could cause new wounds to develop. When these conditions are present, the supervisor must be notified for proper patient care. This tool 30 thus ensures that existing wounds are properly treated and if new wounds develop a cause therefore can more accurately be determined. It will also be understood that while in one embodiment the questions seek “yes” or “no” answers, a variety of other questions could be designed to elicit other responses that still provide the same information. Further, the order of questions is not important and may obviously be varied or reworded. Moreover, more or less questions may also be utilized on the tool 30.

Additionally, the staff member must identify on the tool 30 the patient's name and the date the examination occurred in the respective areas designated by reference numbers 72, 74. The staff member must also sign the record in the area indicated by reference number 76. Further, the supervisor must also sign the tool 30 in the area indicated by reference number 78. This helps ensure that the supervisor is aware of all relevant information and if any remedial measures are not taken, the tool 30 makes it easier to determine who was responsible for any patient neglect. The shift in which the staff member was working at the time of the examination is also reflected in the area indicted by reference number 80.

FIG. 3 is an illustration of an additional portion 90 of the tool 30, such as would be located on the backside or bottom thereof. This additional portion 90 in one embodiment allows the staff member to provide any additional information that may be relevant to the patient's care, in the area generally indicated by reference number 92, such that neglect is prevented and the onset of new skin wounds is prevented. This also requires the signature of the staff member in the area designated by reference number 94.

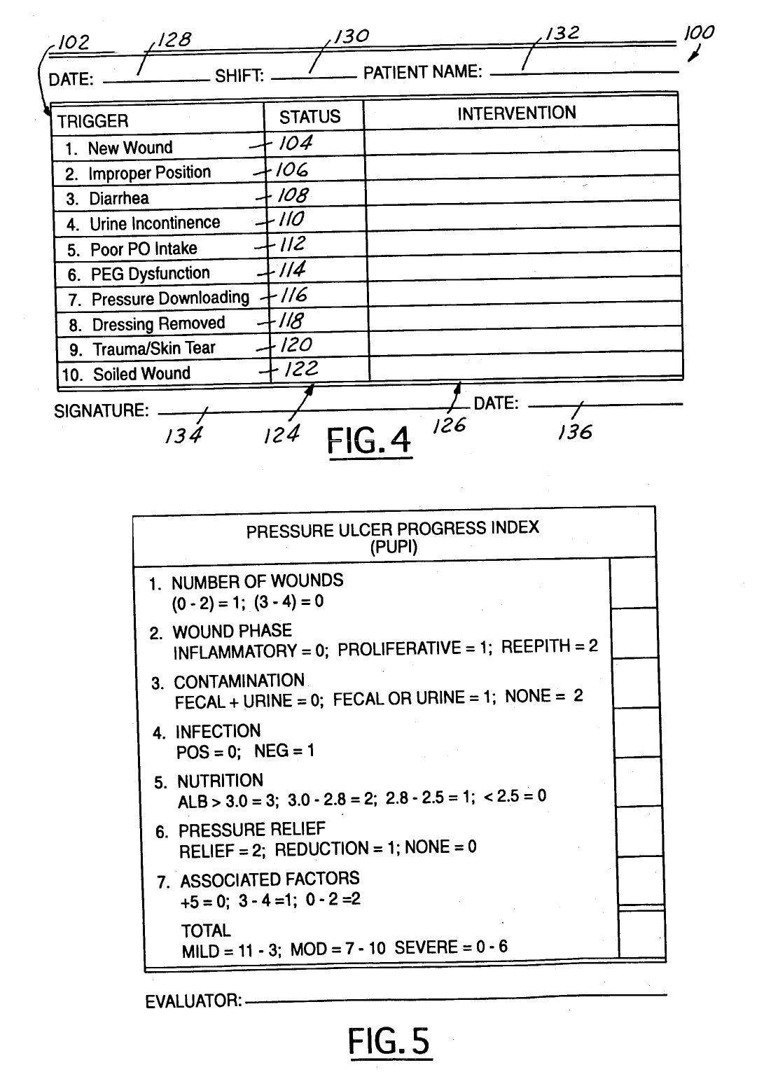

Referring now to FIG. 4 which illustrates an exemplary tool 100 for use in connection with the skin care surveillance system 10. In one embodiment, the exemplary tool 100 is used for oversight of the tool 30 and as a compass guiding the skills development training in the facility as it relates to skin care and skin wounds. The tool 100 is intended to be filled out by the supervisor and is intended as a check of the examination performed by the staff member and reflected on the primary tool 30. The exemplary tool 100 in one embodiment also includes a list of common triggers and is contained in a table format. The table includes a column labeled triggers, as generally indicated by reference number 102, which contains various common triggers for the staff member to look for during the examination.

The first trigger contained in the table is to look for whether a new wound exists on the patient, as generally indicated by reference number 104. The second trigger contained in the table is to determine whether the patient is located in an improper position, as generally indicated by reference number 106, which is a known potential cause for a new wound. The third trigger contained in the table seeks a determination of whether the patient had an onset of diarrhea, as generally indicated by reference number 108, which is a potential cause of the onset of a new wound. The fourth trigger contained in the table is to determine whether the patient was experiencing urine incontinence, as generally indicated by reference number 110, which also is a potential cause for the development of a new wound. The fifth trigger contained in the table is to find out if the patient had a poor oral (PO) intake, as generally indicated by reference number 112, which also is a potential cause for a new wound.

The sixth trigger contained in the table is to determine whether a Gastrostomy tube (PEG) dysfunction occurred, as generally indicated by reference number 114, which also is a potential cause for a new wound. The seventh trigger contained in the table is to determine whether the patient was experiencing any pressure downloading, as generally indicated by reference number 116, which also is a potential cause for a new wound. The eighth trigger contained in the table is to determine whether the patient's dressing was removed, whether by the patient or otherwise, as generally indicated by reference number 118, which can cause the onset of a new wound or affect the healing of an existing wound. The ninth trigger contained in the table is to determine whether the patient had experienced any trauma or skin tear, as generally indicated by reference number 120. The tenth trigger contained in the table is to observe whether the patient's wound is soiled, as generally indicated by reference number 122. It will be understood that while the table of FIG. 4 contains ten triggers, more or less triggers may be utilized to provide skin care surveillance in accordance with the present invention. Moreover, the order of the triggers may also be varied.

As also shown in FIG. 4, the tool 100 includes a column entitled status and another column entitled intervention that are associated with each of the respective triggers. The status column 124 has a series of rows with a respective row being associated with each of the triggers listed in the trigger column 102. The status column 102 is intended to allow the supervisor to indicate the existence or non-existence of the associated trigger such as with a “no” or “yes” indication or a check to indicate that the supervisor looked at the patient to determine whether the trigger was present. The intervention column 126 has a series of rows with a respective row being associated with each of the triggers listed in the trigger column 102. The intervention column 126 is intended to allow the supervisor to note in the record any intervention that was taken in view of the presence of a particular trigger. For example, if the supervisor noted that the patient was located in an improper position, the status column 102 would preferably contain a “yes” indication and the intervention column 126 would, for example, contain a notation as to what position the patient was in and what intervention or measures the staff member took to eliminate this trigger. The same or similar information would also be included on the primary tool 30.

Once the supervisor has completed the examination of the patient, the staff member preferably places the date the examination occurred on the record, as generally by reference number 128, the shift (i.e., 1st, 2nd, or 3rd) on which the staff member performed the examination, as generally indicated by reference number 130, and the patient's name, as generally indicated by reference number 132. Additionally, the supervisor must sign and date the record upon its completion at locations represented respectively by reference numbers 134, 136. It will be appreciated that the tool 100 can take on a variety of different forms and still help in the prevention of skin care wounds as described herein.

A significant benefit of the skin care surveillance system 10, including the tool 30 is that in the event a patient does develop a skin wound the records can be reviewed to help in analyzing whether the wound was due to improper treatment, neglect, or simply due to the deterioration of the patient. Further, because the information regarding who performed the examination as well as the shift on which the examination was performed is contained on the tool 30, the origin of the wound and any responsible staff member can be more readily determined from record information. This will allow for accountability at all levels of staffing.

The exemplary tool 100 is straightforward, relatively simple to use. In sum, the tool 30 is used to identify triggers, that when activated serve as a call to increase the intensity of service for the individual patient involved. The necessary interventions may be short term, or more long lasting, pending which trigger, or set of triggers are activated. The system—unlike the traditional call light—is difficult to ignore. This is because each staff member involved in the care of the patient, directly or indirectly, is held accountable for the patient's condition. Accordingly, no excuse for neglect is available.

The disclosed skin surveillance system 10 will significantly improve the ability to train staff members in skin care surveillance and prevention and ultimately provide better care to all patients. This in turn will provide a significant financial savings for the institutions that employ the systems, including realized savings in the areas of wound care supplies, law suits, regulatory agency fines, and legal fees.

The direct skin care surveillance system helps maximize wound care prevention through a system of increased communication, decreased staff conflicts, and increased teamwork. Further, internal problems related to managerial issues, staffing issues, and issues related to the different shifts are better identified, thus decreasing the possibility of patient neglect or harm. By incorporating the disclosed skin care surveillance system 10 into the current operations of any long care facility, for example, an additional avenue of protection is provided.

FIG. 5 illustrates another tool 140 for use as part of the skin care surveillance system 10. The tool 140 allows the staff member to more accurately keep track of the number of wounds as well as their severity. This helps monitor if a patient's wounds are improving or getting worse, which allows the supervisor to ensure that proper patient care is being given. The tool 140 is preferably also filled out by the staff member or person examining the patient and is kept in the patient's record or file. The tool 140, in one embodiment, includes a plurality of questions that allow the person filling out the form to provide more specific information about a patient's wounds.

The first question on the tool 140 seeks an identification of the number of wounds on the patient, as generally indicated by reference number 142. The second question, as generally indicated by reference number 144, seeks an indication of the phase in which the wound can be categorized, i.e. its severity. For example, the exemplary categories on the tool 140 list the phases as (a) inflammatory; (b) proliferative; and (c) reepith. The third question, as generally indicated by reference number 146, seeks to determine whether the wound has been contaminated and if so, how. The fourth question, as generally indicated by reference number 148, seeks to determine if the wound is infected. The fifth question, as generally indicated by reference number 150, seeks to determine the patient's level of nutrition. The sixth question, as generally indicated by reference number 152, seeks to determine whether pressure on a wound has been relieved. The seventh question, as generally indicated by reference number 154, seeks to assign a numerical value to various other factors related to the care and status of any wounds.

After the questions have been answered a numerical point value can be totaled up in order that the status of the wounds can be generally categorized. For example, as generally indicated by reference number 156, the wounds are exemplary categorized as being mild, moderate, or severe depending upon the answers to questions 1 through 7 above. The tool 140 also includes a location for the signature of the person performing the evaluation, as generally indicated by reference number 158.

Having now fully described the invention, it will be apparent to one of ordinary skill in the art that many changes and modifications can be made thereto without departing from the spirit or scope of the invention as set forth herein.

In the claims:

1. A skin care surveillance method, comprising:
performing an examination of a patient by a staff member;
identifying whether said patient has any new skin wounds or any triggers that can cause the onset of a new skin wound;
documenting the results of said examination, including the existence of any new wounds or any triggers that can cause the onset of a new skin wound;
notifying a supervisor of said staff member about the existence of any new wounds or any triggers; and
recording in a patient's file the date said examination occurred, the name of said patient that was examined, and the name of said staff member that performed said examination.

2. The method of claim 1, further comprising:
documenting any remedial steps taken to eliminate any triggers that were identified during said examination.

3. The method of claim 1, said triggers to be identified during said examination are selected from the group consisting of:
improper patient position; diarrhea; urine incontinence; poor oral intake; PEG dysfunction; pressure downloading; removal of a wound dressing; skin trauma or tear; or a soiled wound.

4. The method of claim 1, further comprising:
obtaining a signature from said supervisor acknowledging being informed of the results of said examination.

5. The method of claim 1, further comprising:
having said supervisor perform an independent examination of said patient to identify the existence of any new skin wound or any triggers.

6. The method of claim 1, wherein said step of documenting the existence of any new wounds includes graphically illustrating the location of said new wounds on said patient.

7. The method of claim 1, further comprising:
evaluating the severity of any new skin wounds based on a variety of criteria.

8. The method of claim 7, wherein said variety of criteria are selected from the group consisting of:
the number of wounds; the phase of each wound; wound contamination; wound infection; patient nutrition; pressure relief; and associated factors.

9. A skin care surveillance system, comprising:
a primary tool to be filled out by a staff member during an examination of a patient to record the results of said examination, said primary tool containing information to direct said staff member to look for any new skin wounds or any triggers that are known to potentially cause the onset of new skin wounds;
a plurality of areas on said primary tool for said staff member to record the results of said examination based on an identification of any new skin wounds or any triggers; and
a location on said primary tool for a supervisor of said staff member to review sign said primary tool attesting to their knowledge of the results of said examination.

10. The system of claim 9, wherein said triggers are selected from the group consisting of:
improper patient position; diarrhea; urine incontinence; poor oral intake; PEG dysfunction; pressure downloading; removal of a wound dressing; skin trauma or tear; or a soiled wound.

11. The system of claim 9, wherein said primary tool further includes a graphic illustration of a human body to allow for the physical illustration of the location of any new wounds.

12. The system of claim 9, wherein said primary tool further includes an area for said staff member to sign and date said primary tool.

13. The system of claim 9, further comprising:
a secondary tool that is intended to be filled out by said supervisor.

14. The system of claim 13, wherein said secondary tool includes a list of a plurality of triggers to be looked for as said supervisor review said patient and an associated location to allow for the recordation of any remedial steps taken in response to an identification of one or more of said plurality of triggers.

15. The system of claim 9, further comprising:
a wound evaluation tool that allows the severity of any wounds to be evaluated based on predetermined criteria.

16. The system of claim 15, wherein said predetermined criteria are selected from the group consisting of:
the number of wounds; the phase of each wound; wound contamination; wound infection; patient nutrition; pressure relief; and associated factors.

17. A wound care surveillance system, comprising:
a primary tool to be filled out by a staff member to allow for the recordation of the existence of any new wounds or any triggers that are known to potentially cause new wounds to develop; and
a secondary tool to be filled out by a supervisor of said staff member to allow for the recordation of any remedial steps taken in response to the identification of any new wounds or any triggers.

18. The system of claim 17, wherein the triggers to be identified by said staff member are selected from the group consisting of:
improper patient position; diarrhea; urine incontinence; poor oral intake; PEG dysfunction; pressure downloading; removal of a wound dressing; skin trauma or tear; or a soiled wound.

19. The system of claim 17, wherein said primary tool contains a plurality of questions designed to elicit responses from said staff member that will provide an accurate assessment of said patient's condition as of the time of said examination.

20. The system of claim 19, wherein said primary tool includes the following:
a location for said staff member to sign and date said primary tool upon completion of said examination; and
a location for said supervisor to sign said primary tool after being informed of the results of said examination.
